(19) United States
(12) Patent Application Publication
(10) Pub. No.: US 2014/0357985 A1
(43) Pub. Date: Dec. 4, 2014

(54) METHOD FOR VISUAL ASSISTANCE WHEN FIXING AN IMPLANT, AND TARGET APPARATUS

(71) Applicant: SIEMENS AKTIENGESELLSCHAFT, MUENCHEN (DE)

(72) Inventors: JUAN CARDELINO, MONTEVIDEO (UY); ADRIAN EGLI, SURSEE (CH); ALBERTO FERNANDEZ, MONTEVIDEO (UY); ADRIAN JOHN, KAISTEN (CH); GERHARD KLEINSZIG, FORCHHEIM (DE)

(73) Assignee: SIEMENS AKTIENGESELLSCHAFT, MUENCHEN (DE)

(21) Appl. No.: 14/293,106

(22) Filed: Jun. 2, 2014

(30) Foreign Application Priority Data
May 31, 2013 (DE) 102013210185.6

(51) Int. Cl. A61B 17/17; A61B 19/00; A61B 6/12

(52) U.S. Cl. CPC A61B 17/1703; A61B 6/12; A61B 19/5244; A61B 2019/5238
USPC 600/424; 606/97

(57) ABSTRACT

A user is visually assisted when fixing an implant with a locking element in, or on, a bone. The location of a guide instrument for guiding the locking element relative to the implant is set by a target apparatus. The implant can be rotated and/or positioned by moving the target apparatus. The guide instrument carries at least one x-ray marker. An x-ray device records an x-ray image of the target region of the locking element and the x-ray marker. The orientation and position of a projected straight line, which forms a projection of a longitudinal axis of the guide instrument in the image plane, is determined from the position and/or form of the image of the x-ray marker. A superposed illustration is calculated of the x-ray image with a graphical element indicating the orientation and position of the projected straight line and the superposed illustration is output on a display.

### CROSS-REFERENCE TO RELATED APPLICATION

This application claims the priority, under 35 U.S.C. §119, of German patent application DE 10 2013 210 185.6, filed May 31, 2013; the prior application is herewith incorporated by reference in its entirety.

### BACKGROUND OF THE INVENTION

1. Field of the Invention

The invention relates to a method for visually assisting a user when fixing an implant by way of a locking element in, or on, a bone. The location of a guide instrument for guiding the locking element in relation to the implant is set by a target apparatus. The implant can be rotated and/or positioned by moving the target apparatus. The invention also relates to a target apparatus.

Bone fractures are often treated by the introduction or application of supporting implants into/on the bone. An advantage of using implants when treating bone fractures is that, with the aid of these implants, a load can quickly be applied onto the bone again. Intramedullary rods, in particular, allow the patient to be mobile again within a very short period of time, even in the case of fractures on supporting bones, such as the femur. This reduces the period of being bedridden and a multiplicity of complications are avoided. In order to fasten such an implant in, or on, the bone, locking elements (also referred to as fastening elements), such as e.g. screws, are guided through the implant and fastened to the bone. In order to enable fastening that is as stable as possible, it is advantageous to use fastening elements that are as long as possible. At the same time, it is necessary to prevent the fastening elements from penetrating too far through the bone and from entering the tissue or joints. In order to achieve a longer fastening length, at least one part of the fastening elements is often arranged in regions of the bone which are angled. Thus, for example, when introducing an intramedullary rod into the femur, the neck or head of the femur is often also used for fastening the intramedullary rod.

In such cases, it is essential to position the implant and the locking elements thereof in such a way that the locking elements penetrate into the corresponding angled part of the bone. In this respect, a method is often employed, in which, initially, a Kirschner wire (K-wire) is introduced into the bone at the position and at the angle at which the introduction of a locking element is envisaged. The position of the Kirschner wire can be checked in subsequent x-ray recordings and, if necessary, be adapted. The Kirschner wire is only replaced by the locking element after the Kirschner wire is in a position in which the locking element is intended to be arranged.

This method is disadvantageous since the Kirschner wire often needs to be introduced into the bone, and removed from it again, a multiple number of times. However, the bone is damaged by the introduction and removal of the Kirschner wire. Firstly, this can have a negative effect on the healing of the bone and, secondly, there is the risk of the stable hold of a locking element in the bone being impaired by the destruction of the bone structure.

Furthermore, what is disadvantageous is that a plurality of x-ray recordings are required in this method since at least one, but often two or more, x-ray recordings are made after each introduction of the Kirschner wire.

A further problem in the above-described method is that Kirschner wires are more flexible than the locking elements to be introduced. Therefore, it is possible that the Kirschner wire bends during the introduction into the bone. This leads to the position of the Kirschner wire in the bone not necessarily being identical to the position and orientation of a locking element which is introduced in place of the Kirschner wire.

### SUMMARY OF THE INVENTION

It is accordingly an object of the invention to provide a a method for assisting a user when fixing an implant and a corresponding targeting apparatus which overcome the disadvantages of the heretofore-known devices of this general type and which provides for a method that leads to a reduction in the damage to the bone structure and, possibly, to a reduction in the radiation exposure and to an improvement of the fastening of the implant.

With the foregoing and other objects in view there is provided, in accordance with the invention, a method for visually assisting a user when fixing an implant by way of a locking element in or on a bone, the method comprising:
providing a guide instrument with at least one x-ray marker;
setting a location of the guide instrument for guiding the locking element in relation to the implant with a target apparatus, wherein the implant can be rotated and/or positioned by moving the target apparatus;
recording at least one x-ray image within a defined image region containing the target region of the locking element and the x-ray marker;
determining an orientation and a position of a projected straight line, which forms a projection of a longitudinal axis of the guide instrument in an image plane, from at least one of a position or a form of the image of the x-ray marker in the x-ray image using a computer;
calculating with the computer a superposed illustration of the x-ray image with a graphical element indicating the orientation and position of the projected straight line; and
outputting the superposed illustration on a display apparatus.

In other words, the objects of the invention are achieved by providing a guide instrument with at least one x-ray marker and carrying out the following steps: recording at least one x-ray image, the image region comprising the target region of the locking element and the x-ray marker, by means of an x-ray device, establishing the orientation and position of a projected straight line, which forms a projection of a longitudinal axis of the guide instrument in the image plane, from at least the position and/or form of the image of the x-ray marker in the x-ray recording using a computer, calculating a superposed illustration of the x-ray image with a graphical element indicating the orientation and position of the projected straight line by means of the computer, and outputting the superposed illustration on a display apparatus.

The concept underlying the invention is that the Kirschner wire in the method set forth at the outset serves to mark an end position of the locking element in the x-ray image. In the method according to the invention, the Kirschner wire is intended to be replaced by a virtual Kirschner wire. In the method set forth at the outset, the Kirschner wire is normally introduced with the aid of a guide instrument, in particular a tissue protection sleeve. This guide instrument has a plurality of objects. Firstly, the tissue is intended to be protected when introducing the Kirschner wire or the locking element and, secondly, the Kirschner wire or the locking element is intended to be guided in a straight line. Thus, in the ideal case, the Kirschner wire in the bone constitutes an extension of the guide instrument.

In the simplest case, only a position and orientation of the virtual Kirschner wire in the image plane, i.e. a projected straight line of the longitudinal axis of the guide instrument, is intended to be established in the method according to the invention. With the aid of the projected straight line, it is possible to obtain all items of information which can also be obtained in the conventional method by the use of a Kirschner wire. In order to establish the projected straight lines, it is necessary, in an x-ray recording, to establish the position and orientation of the guide instrument in the image plane of the x-ray recording. In the method according to the invention, this is achieved by virtue of at least one x-ray marker on the guide instrument being used. A projected straight line, which represents the projection of the longitudinal axis of the guide instrument in the image plane, or which extends the longitudinal axis, can subsequently be established from the position and/or form of the x-ray marker or x-ray markers. Using this information, a superposed illustration of the x-ray image with a graphical element, which indicates the location of this projected straight line, can be generated and depicted. However, an image which simultaneously shows the x-ray image and an extension of the projection of the longitudinal axis of the guide instrument provides the same visual information (and in embodiments of the invention also additional visual information) as an image which is generated by virtue of a Kirschner wire being introduced into the bone with the aid of the guide instrument and an x-ray recording subsequently being generated.

The method is suitable for a multiplicity of locking elements. Thus, it is also possible to use the method when only a Kirschner wire is to be introduced, and the Kirschner wire is not intended to be replaced by a different locking element. However, it can also be used for screws, clamps or the like. In particular, the method is suitable for locking intramedullary rods. Locking intramedullary rods is particularly challenging since, in this case, the implant is situated within the bone and therefore no direct access to the implant is possible during fastening. When introducing intramedullary rods, use is often made of target apparatuses, which substantially consist of a bracket which is connected to the intramedullary rod in a detachable but positionally and rotationally secured manner and on which a guide instrument or a fastener for a guide instrument is provided. With the aid of such a target apparatus, it is usually possible to move an implant along an axis of the bone or to rotate the implant about this axis. However, at the same time, such a target apparatus ensures that locking elements which are introduced into the implant with the aid of a guide instrument arranged on the target apparatus hit a predetermined position, i.e., in general, a passage opening of the implant for the locking element.

If only one x-ray recording is recorded in the method, it is not possible without additional information to make statements about the form and arrangement of the bone in three dimensions. This is particularly problematic since it is often desired to introduce locking elements into parts of the bone which are not rotationally symmetric. Therefore, in order to obtain more information about form and structure of the bone, and to obtain additional information about the orientation of the target instrument, it is advantageous if at least two x-ray images are recorded offset to one another, either using different projection directions or with parallel projection directions and the recording being perpendicular to the projection direction. In the simplest case, two x-ray recordings are recorded in such a way that the image planes of the recordings are perpendicular or virtually perpendicular to one another. In this case, it is possible to obtain information about the extent of the bone in all three spatial directions and information about the orientation of the guide instrument and hence about the orientation of an introduced locking element. On the other hand, it is often complicated during surgery to record x-ray recordings in two orthogonal directions since the examination object needs to be accessible from two sides in this case. Therefore, it can be advantageous to record the x-ray recordings at flatter angles with respect to one another. Thus, for example, the angle between the projection directions of the two recordings may be 45°. However, it is also possible to record the recording with a smaller or larger angle between the projection directions, for example 5° or 30° on the one hand, or 60° or 80° on the other. Alternatively, it may be advantageous in an x-ray device with cone beam geometry to record the x-ray recordings in the same direction, but to displace the recording point within the image plane. The advantage thereof is that three-dimensional image information can also be obtained in this case and, at the same time, recording is very simple since there only needs to be a displacement within one plane of the x-ray device or, at least, of the recording arrangement. At the same time, the x-ray images can easily be interpreted in this case since they were recorded from an angle which a user is used to.

The two x-ray images can be used to display a superposed illustration with the longitudinal axis, projected in the corresponding plane of the x-ray recording, of the guide instrument for each one of the two x-ray images, as described above. As a result, the user obtains unambiguous visual information relating to the position of the bone at which a locking element introduced in this position of the target apparatus would be situated, and the user can moreover estimate how long such a locking element may be, in particular if the size calibration of the x-ray images is known.

It is moreover possible that the position and orientation of the longitudinal axis of the guide instrument is calculated, wherein, in particular, known limitations in the degrees of freedom of movement of the target apparatus and/or the form of the image of the x-ray marker in the x-ray image and/or the relative position of at least two x-ray markers in the x-ray image and/or at least two x-ray images are employed for determining the angle between the image plane of the x-ray image and the longitudinal axis, and for calculating the position of the longitudinal axis.

Determining the location of the longitudinal axis of the guide instrument is particularly simple if two or more x-ray images are recorded and, in particular, the projection directions of the x-ray images are very different. In this case, the location of the longitudinal axis is uniquely determined by the two known projected straight lines. Such a calculation is also possible for small angles between the projection directions or for a displacement between the x-ray images in an image plane, wherein the resolution capability in one of the spatial directions is significantly reduced for small angles or displacements.

However, recording a plurality of x-ray images leads to higher radiation exposure of the patient. However, in many cases, it is also possible to determine the projection and orientation of the longitudinal axis of the guide instrument from a single x-ray image. By way of example, if two x-ray markers are arranged on the guide instrument in the direction of the longitudinal axis of the guide instrument, it is possible to determine an angle between guide instrument and image plane from the distance between these x-ray markers if the size calibration of the image is known. If more than two markers are used, such a determination can also be achieved independently of the size calibration, for example by virtue of a plurality of markers being arranged perpendicular to the longitudinal axis and along the longitudinal axis. By way of example, three x-ray markers can be arranged around the circumference of a round guide instrument and a further x-ray marker can be offset along the longitudinal axis. From the relative distances of the first three x-ray markers, it is possible to determine a rotational angle of the guide instrument about the longitudinal axis, a scale from the absolute distances and the rotational angle of these three points, and an angle between longitudinal axis and image plane from the distance to the fourth point along the longitudinal axis. Naturally, a multiplicity of further arrangements of x-ray markers is possible.

It is likewise possible to determine an angle between image plane and guide instrument or a relative position with respect to the image plane by the form of an x-ray marker or by using a plurality of x-ray markers with different forms.

In many cases, it is moreover possible to use known restrictions of the degrees of freedom of movement of the guide instrument for establishing the position and orientation of the longitudinal axis from a single x-ray image. Thus, as set forth at the outset, it is usual when fastening an intramedullary rod for the latter to be guided within the bone and therefore the rod can, with the aid of the target apparatus, only be displaced along, or rotated about, an axis of the bone into which it was introduced. A displacement of the intramedullary rod along the axis of the bone in this case leads to a displacement of the x-ray marker along the bone axis. A rotation of the intramedullary rod leads to a movement of the markers perpendicular to this direction. Therefore, in this case, the orientation of the guide instrument with respect to the image plane can be determined completely using a single x-ray marker and a single x-ray image.

In order to arrive at a more robust method, it is often advantageous to combine a plurality of the aforementioned options for determining the position and orientation of the longitudinal axis of the guide instrument.

Determining the position and orientation of the longitudinal axis of the guide instrument is advantageous then, in particular, if, after recording the x-ray image, a bone model is calculated by the computer using the x-ray image, wherein the bone model is generated, in particular, from a parameterized model data record, which is adapted to the bone, or wherein the bone model is calculated, in particular, by registering the x-ray image with an anatomical atlas.

In this case, both three-dimensional position and orientation data of the guide instrument and three-dimensional dimensions of the bone can be obtained using the method according to the invention. By way of example, using these data it is possible to determine the final position of a predetermined locking element which is introduced into the bone with the current position and orientation of the guide instrument. In particular, it is possible to determine whether the locking element is seated centrally in a bone or on the edge thereof, and whether the locking element projects beyond the bone. Naturally, these data can also be used to optimize the position of the implant for better locking purposes or it is possible to determine parameters of the locking elements.

In particular, the bone models can be determined from a single x-ray image. By way of example, this is possible if an anatomical atlas which contains 3D data of the bones is present. In this case, with the x-ray image, there can be a 2D/3D registration with the data of the anatomical atlas and it is possible to select a fitting 3D data record which corresponds to the examined bone to the best possible extent from the anatomical atlas. However, alternatively, it is also possible to determine 3D data from the two-dimensional x-ray image by virtue of a parameterized model of the imaged bone being fitted to the x-ray image by optimizing the parameters.

After calculating the bone model and the position and orientation of the longitudinal axis, it is possible, in particular, that at least one parameter of the locking element is calculated, wherein, in particular, a locking element is selected from a group of possible locking elements and/or a length of the locking element is determined, and/or a position correction between a current position and an intended position of the target apparatus is calculated, wherein the position correction, in particular, describes a rotation of the target apparatus about an axis extending through the bone and/or a shift between target apparatus and bone along this axis.

Implants can usually be locked with a multiplicity of locking elements which, in particular, differ in terms of their length, but possibly also differ in terms of further parameters, such as e.g. a thread pitch or a head shape. Since it is possible in the method according to the invention to determine a complete 3D model of the bone, into which the locking elements are introduced, and a three-dimensional position and orientation of the guide instrument, it is easy to establish individual parameters of the locking elements. Thus, for example, the maximum length of the locking element can be determined from a predetermined minimum distance from a bone surface for a currently set position of the guide instrument. If a database with available or obtainable locking elements is available, it is possible to select from this database the locking element with the best fit or a locking element which has a certain maximum or minimum value for a parameter.

However, additionally or alternatively, it is also possible to determine with the method according to the invention that the current position of the guide instrument is not ideal. Although it is possible in this case to propose a locking element which can nevertheless be used, as described above, it is however advantageous to suggest a position correction of the implant to the user such that it is subsequently possible to use a longer or more suitable locking element. However, other parameters which may be optimized are also conceivable. Thus, it may be possible to set the angle between locking element and implant. This can be brought about continuously, or else with predetermined steps.

The additional information can be made accessible to a user in multifaceted ways. It is possible that, in addition to the superposed illustration on the output instrument, an alphanumerical or graphical item of information is displayed, which represents the calculated parameter of the locking element and/or the position correction. Thus, for example, there can be an alphanumerical display of the type of the ideally to be used locking element. However, it is also possible to indicate that it would be advantageous to move the target apparatus in a certain direction or to rotate it about a specific axis. However, a graphical illustration is often more intuitive for a user. Thus, for example, it is possible to show an animation which shows a movement of the target apparatus from an actual position to an intended position, or it is possible to show, using different colors, an ideal locking element for the actual position and an ideal locking element for an ideal position. In the superposed illustration, it is also possible to specify position corrections by arrows, or the like. In particular, such a display can also be interactive. That is to say that, for example, a freely rotatable bone model can be depicted, in which, for example, a position of a locking element which would be introduced with the current position and orientation of the longitudinal axis of the guide instrument can be displayed. It is also possible to indicate, simultaneously or alternatively, an illustration in which the implant is displaced in a purely virtual manner or in which the locking element is introduced differently or replaced in a purely virtual manner. Using this, a user can interactively establish the possible effects of different interventions.

In addition to purely showing notifications, it is also possible to instruct a user of the method directly. Thus, it is possible, depending on the calculated position correction, for the user to be provided with optical, acoustic or haptic notifications for correcting the position of the target apparatus. Optical notifications can be shown directly on the display apparatus; however, it is also possible that the target apparatus itself has a display or other display elements, which can provide the user with notifications in respect of a position correction. Alternatively or additionally, the user can also be provided with acoustic notifications. By way of example, a regular noise can become faster or slower when the target apparatus and hence the end position of the locking element to be introduced approach or retreat from an ideal position. Alternatively or additionally, there can also be speech output which provides the user with notifications as to how the position of the target apparatus can be improved. A multiplicity of further acoustic signals are also possible. Moreover, the user can be provided with haptic notifications. Thus, for example, a part of the target apparatus can vibrate or carry out a directed movement in order to provide the user with notifications in relation to an advantageous position correction. Naturally, the haptic notifications can also be used by separate devices which are worn on the body of the user or which are arranged in a region in contact with the latter.

It is also possible for the graphical element to depict the calculated parameter of the locking element, in particular by displaying an image of a locking element, which has this parameter, in the locking position. By way of example, a locking element ideal for the current position and orientation of the longitudinal axis can be selected and the selected locking element can be depicted in the position in which it would be fastened if it were to be attached in the case of the current position and orientation of the longitudinal axis. Precisely such an illustration is also possible for the ideal position and orientation of the longitudinal axis. It is also possible for the user to be able to select whether the locking element for the current position and orientation, the ideal position and orientation, or both, are displayed for him. Particularly if a bone model is available, it is also possible for this illustration to be freely rotatable. It is moreover possible for the user to modify parameters established within the scope of the method or for the user to independently select parameters not established within the method.

It is always desirable to reduce the radiation exposure of a patient. A substantial advantage of the method according to the invention over the conventional search for the ideal position of a locking element by repeated introduction of a Kirschner wire lies in the fact that a bone model established once can always be used again. It is therefore possible, after establishing the orientation and position of the projected straight line or of the longitudinal axis, for the position and/or orientation of the target apparatus or of the guide instrument to be detected continuously by a second detection system, in particular an optical detection system, and for the orientation and position of the projected straight line or of the longitudinal axis to be updated continuously on the basis of this information.

By way of example, it is possible that a bone model is obtained from a first x-ray recording by using an anatomical atlas, that a position and orientation of the guide instrument in the three-dimensional space is determined by using further information from the single x-ray recording, as described at the outset, and that a position correction is determined from these two items of information. If only x-ray images are used for determining the position of the guide instrument, a further x-ray image would now have to be recorded after each correction by the user. However, as described, a further detection system can advantageously be used here for continuously updating the position and orientation of the longitudinal axis. As a result, it is possible to provide the user continuously with current information and position corrections. By way of example, it is possible only to record a second x-ray image when, in accordance with the information from the second detection system, the position is ideal. Using this, the same reliability continues to be achieved as in the case of complete positioning with the aid of x-ray images, but the radiation dose of the patient is significantly reduced in this case.

For positioning implants, use is often made of x-ray devices with a restricted field of view, in particular since the radiation exposure for the examination object is also reduced when the field of view is restricted. Therefore, it is advantageous if the x-ray marker is arranged on the end of the guide instrument facing the implant.

With the above and other objects in view there is also provided, in accordance with the invention, a target apparatus for assisting a user when fixing an implant by way of a locking element in or on a bone, the target apparatus comprising:
a fastening element for fastening the target apparatus to the implant in a spatially secured manner;
a guide instrument for guiding the locking element, the guide instrument having at least one x-ray marker; and
a connection element for coupling the fastening element and the guide instrument in a spatially secured manner.

In other words, there is provided a target apparatus for assisting a user when fixing an implant, in particular when carrying out the method according to the invention, by means of a locking element in, or on, a bone, comprising a fastening element for fastening the target apparatus to the implant in a spatially secured manner, a guide instrument for guiding the locking element and a connection element for coupling the fastening element and the guide instrument in a spatially secured manner, which target apparatus is distinguished in that the guide instrument has at least one x-ray marker. The target apparatus according to the invention can be used in all described embodiments of the method according to the invention.

Here, it is particularly advantageous if the guide instrument is a tissue protection sleeve for protecting the tissue when introducing the locking element. Such tissue protection sleeves are used in any case in a multiplicity of methods for locking an implant. Therefore, it is possible with very little outlay to reequip available devices for use in a method according to the invention.

Moreover, it is advantageous if the x-ray marker is arranged at the end of the guide instrument facing the implant.

Other features which are considered as characteristic for the invention are set forth in the appended claims.

Although the invention is illustrated and described herein as embodied in a method for visual assistance when fixing an implant, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 shows a flowchart of an exemplary embodiment of the method according to the invention,

FIG. 2 shows a flowchart of a further exemplary embodiment of the method according to the invention,

FIG. 3 shows an exemplary embodiment of a target apparatus according to the invention, attached to an intramedullary rod,

FIG. 4 schematically shows an x-ray image recorded in a method according to the invention,

FIG. 5 schematically shows a superposed illustration of the x-ray image from FIG. 4 with a projected straight line,

FIG. 6 schematically shows a superposed illustration of the x-ray image from FIG. 4 with a virtual locking element, and

FIG. 7 schematically shows a superposed illustration of the x-ray image from FIG. 4 with the illustration of a projected straight line, an alphanumerical display of a parameter and a display of a position correction.

### DETAILED DESCRIPTION OF THE INVENTION

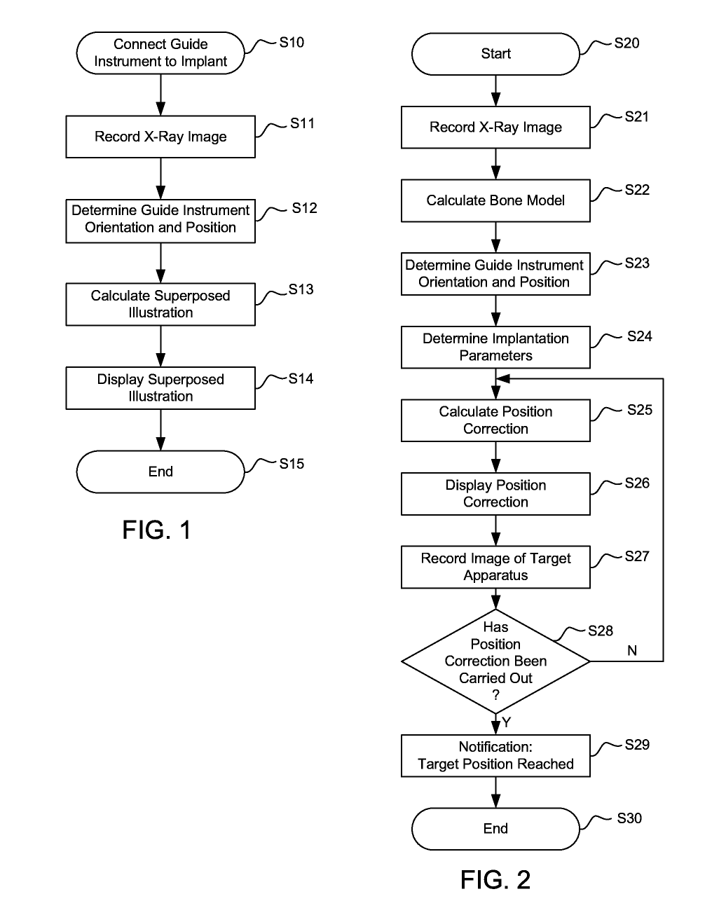

Referring now to the figures of the drawing in detail and, more specifically, to FIG. 1 thereof, there is shown a flowchart of an exemplary embodiment of the method according to the invention. At the start of the method in step S10, an implant has already been introduced into a patient and a guide instrument for guiding a locking element for the purposes of fixing the implant by means of a target apparatus is connected in a spatially secured manner to the implant.

In step S11, an x-ray image is initially recorded, wherein the image region of the x-ray image comprises the target region of the locking element, i.e. the region into which the locking element is intended to be introduced, and an x-ray marker which is attached to the guide instrument. The x-ray image is recorded using a conventional x-ray device, wherein, for example, an individual recording can be recorded in the anterior/posterior direction. A plurality of x-ray images can also be recorded during this step, which x-ray images can then be used in the further method steps, in particular for obtaining three-dimensional spatial, orientation and extent information relating to the guide instrument and/or at least one bone. To the extent that the further description refers to the recorded x-ray image, the use of a number of x-ray images recorded in this step is always possible.

Subsequently, in step S12, the orientation and position of the projected straight line which forms the projection of a longitudinal axis of the guide instrument in the image plane is determined. Establishing the orientation and position of the projected straight line is brought about by identifying the x-ray markers and examining the position and/or form of the image of the x-ray markers in a computer. For the purposes of identifying the x-ray markers, it is possible to use conventional image processing algorithms, for example edge detection. Detecting the position and orientation of the projected straight line is then possible in a particularly simple manner when at least two x-ray markings are arranged on the guide instrument or when the x-ray marker has an elongate form. Depending on the type of implant and the degrees of freedom of movement of the implant resulting there from, a single x-ray marker may, however, also be sufficient for determining the position and orientation of the projected straight line. Thus, for example, an intramedullary rod can clearly be identified in the x-ray image and the longitudinal axis of the intramedullary rod can easily be determined. Since the intramedullary rod is situated in the interior of the bone all that is possible is a movement of the intramedullary rod along this axis and a rotation of the intramedullary rod about this axis. However, the target apparatus is rigidly connected to the intramedullary rod and the guide instrument is in turn rigidly connected to the target apparatus. The movement restrictions for the intramedullary rod thus lead to movement restrictions for the guide instrument, as a result of which the movement axis thereof is predefined. Thus, in this case, it is sufficient to establish the position of the x-ray marker along the bone in order to unambiguously set the position and orientation of the projected straight line.

In step S13, a superposed illustration of the x-ray image with a graphical element indicating the orientation and position of the projected straight line is calculated. A particularly simple representation of the position and orientation of the projected straight line is a line which is superposed on the x-ray image. Here, the superposed line can, for example, be shown using a different color than the x-ray image. However, the graphical element can also be a representation of a locking element. By way of example, the parameters of this locking element can be entered by the user in advance. However, it is also possible for further steps to be integrated into the method in order to establish these parameters.

The representation calculated in step S13 is depicted in step S14 on a display apparatus, whereupon the method ends with step S15. Thus, a user is already provided in this simplest embodiment of the method with the same information as in the case of a method in which the positioning of an implant is brought about with the aid of a trial-and-error principle and the repeated introduction of a Kirschner wire. At the same time the damage to the bone structure, which usually occurs in such a method, is avoided.

FIG. 2 shows a flowchart of a further exemplary embodiment of a method according to the invention. Compared to the method depicted in FIG. 1, additional steps are integrated into the method in this case, as a result of which additional information is made available to the user and continuous assistance in finding the ideal implant position is made possible.

The preparatory steps before the method starts in step S20 and the recording of the x-ray image in step S21 take place analogously to the method explained with reference to FIG. 1. However, in this method, a bone model is calculated in step S22 from the x-ray image recorded in step S21. The bone model is calculated by using an anatomical atlas. To this end, the computer carries out a 2D/3D registration of the x-ray image with a multiplicity of three-dimensional image data records, stored in a database, of the bone type in which the implant is intended to be locked. The methods for 2D/3D registration and for selecting the fitting data record are known from the prior art; therefore, these shall only be discussed briefly and in an exemplary manner here. By way of example, the registration can be carried out by virtue of projection images being calculated from the 3D data records, which projection images are subsequently registered with the x-ray image. A multiplicity of registration methods enable simultaneous specification of a value describing the quality of the registration. In this case, it is possible to select the 3D data record whose projection image can be registered best to the x-ray image.

Alternatively, it would be possible in this step to use a bone model which is parameterized by the two-dimensional image data of the x-ray image. By way of example, if it is known that a bone only differs in different examination objects by virtue of this difference being parameterizable by a few parameters, which can all be gathered from a two-dimensional recording, it is also possible to directly determine a three-dimensional bone model from the 2D x-ray image.

Independently of the selected method for generating the bone model, a three-dimensional model of the bone is available after step S22, into or onto which bone the implant is intended to be locked. Complementing this information, the position and orientation of the longitudinal axis of the guide instrument is determined in step S23. This is then possible in a particularly simple manner if the implant is an intramedullary rod, i.e. if it is elongate and arranged in a bone. Intramedullary rods are used e.g. for treating fractures of femurs, shinbones or humeri. When an intramedullary rod is introduced, the implant can only be displaced along the axis of the bone and rotated about this axis. Using such a limitation of the degrees of freedom of movement, it is already possible to identify the orientation and position of the longitudinal axis by segmenting and evaluating an individual x-ray marking. If an implant is arranged with more degrees of freedom, or if redundancy in the data is to be achieved, it is advantageous to arrange a plurality of x-ray markings on the guide instrument or to use x-ray markings with specific forms, such as e.g. elongate x-ray markings. Hence, it is possible to determine the orientation of the longitudinal axis of the guide instrument using known geometric calculations by evaluating the position and/or form of the x-ray marking, in particular taking into account the possible degrees of freedom of movement, or from the relative positions of the plurality of markings.

Since now both a three-dimensional bone model and information relating to the position or orientation of the longitudinal axis are available, implantation parameters can be determined in step S24. Hence, it is possible to establish a maximum length of the locking element that can be introduced at the current position and with the current orientation of the guide instrument, without penetrating through the bone and entering a joint or the tissue. Moreover, it is possible to determine whether the current position and orientation of the guide instrument is well-suited or badly suited for the introduction of locking elements. Thus, what is normally intended to be achieved is that the locking element is surrounded uniformly on all sides by the bone. Moreover, it is usually advantageous, if this is possible, to employ longer locking elements. If it is established that it is possible through a displacement or a rotation to obtain a positioning of the locking element which, according to these facets, is better than the position to which the current position/orientation of the guide instrument would lead, it is possible to establish a better position and orientation of the guide instrument.

If it was established in step S24 that a better position and/or orientation of the guide instrument are possible, a position correction is calculated in step S25. For an intramedullary rod, such a position correction can consist of a displacement distance along the longitudinal axis of the bone, into which the intramedullary rod has been introduced, and a rotational angle about the longitudinal axis of this bone. In step S26, the position correction is displayed on a monitor for a user. The display is brought about as a superposed display on a sectional illustration of the bone model. Arrows which indicate a length or an angle, along or about which the target apparatus is to be displaced or rotated, are superposed onto said sectional illustration.

After outputting the proposed position correction, an image of the target apparatus is recorded in step S27 with the aid of a video detection system. Said target apparatus has optical markings which enable the optical system to determine the change in the position and orientation of the target apparatus. After recording the video image, a check is carried out in step S28 as to whether the proposed position correction has already been carried out. If the position correction has not yet been carried out, the method is repeated from step S25 onward and a new position correction is calculated. However, when calculating the position correction, a movement or rotation of the target apparatus detected by the video camera is registered and included in the calculation of the new position correction. Hence, continuous updating of the position correction is possible by continuous updating of the established position and orientation of the longitudinal axis of the guide instrument. The method steps from step S25—an updating of the position correction—to step S28—the querying as to whether the target position has been reached—are repeated until the target position has been reached. A notification for the user is emitted in step S29 as soon as the target position has been reached, i.e. as soon as the actual position of the guide instrument corresponds to the ideal position of the guide instrument, as established in step S24, for introducing a locking element. Therewith, the method ends in step S30.

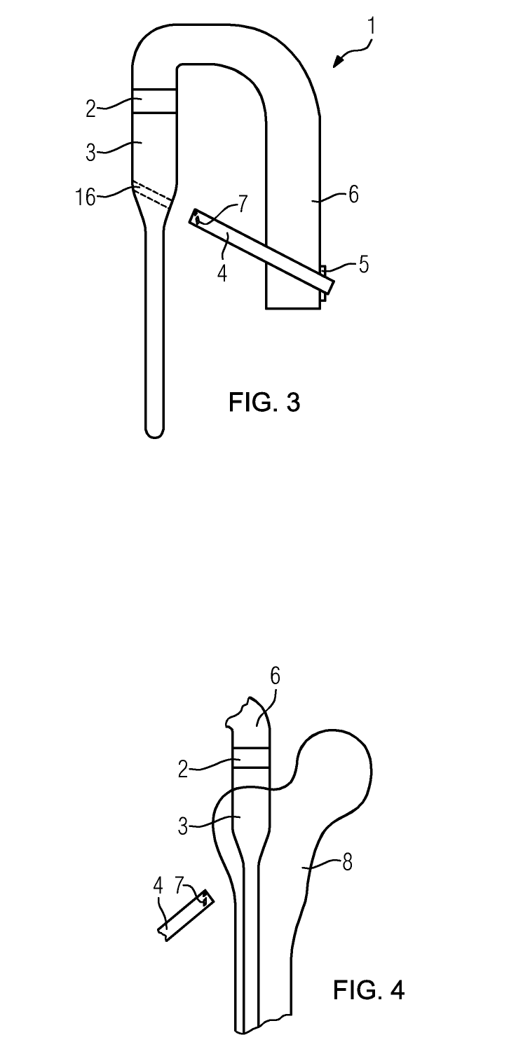

FIG. 3 shows a target apparatus according to the invention. The target apparatus 1 is connected to the implant 3, in this case an intramedullary rod, by means of a fastening element 2. The connection is rigid and secured against rotation. Moreover, the target apparatus 1 has a guide instrument 4. The guide instrument 4 substantially consists of a hollow tube, which guides a Kirschner wire or a locking element into an opening 16, embodied for holding the locking element, of the implant 3. The guide instrument 4 is embodied as a tissue protection sleeve. Therewith, the guide instrument 4, which can be fastened by a screw 5 or can be displaceably mounted, becomes introducible into the tissue. This prevents the locking element from coming into contact with the tissue during the introduction into the bone. The guide instrument 4 is secured on the connection element 6 by the screw 5, as a result of which a spatially secured arrangement with respect to the implant 3 is achieved. In order to be able to identify the position and orientation of the guide instrument 4 in an x-ray image, x-ray markers 7 are arranged on the guide instrument 4. Said x-ray markers are situated in the region facing the implant 3. The guide instrument 4 itself consists of a material which is almost completely transmissive to x-rays, for example a plastic such as PET, PVC or Teflon. Alternatively, the guide instrument 4 can also consist of a carbon fiber composite. The x-ray markers 7 are made of a strongly x-ray-attenuating material, in particular a metal such as steel, aluminum or titanium. The x-ray markers 7 can be spheres, rings or cuboids. The degrees of freedom of movement of the guide instrument 4 are greatly limited when introducing an intramedullary rod into a bone. Therefore, it is already possible, in a robust manner, to determine location and orientation of the guide instrument 4 using the two x-ray markers 7 attached to the guide instrument.

FIG. 4 shows an example of an x-ray image, as it is recorded at the beginning of the method. The x-ray image shows a bone 8, into which the intramedullary rod 3 should be locked. The intramedullary rod 3 is fastened to the target apparatus 6 by the fastening element 2. The guide instrument 4 is likewise fastened to the target apparatus 6, with this connection not being visible in the image as a result of it being situated outside of the field of view of the x-ray device. The two x-ray markers 7 can clearly be identified on the guide instrument 4, which can itself only be weakly identified in the x-ray image.

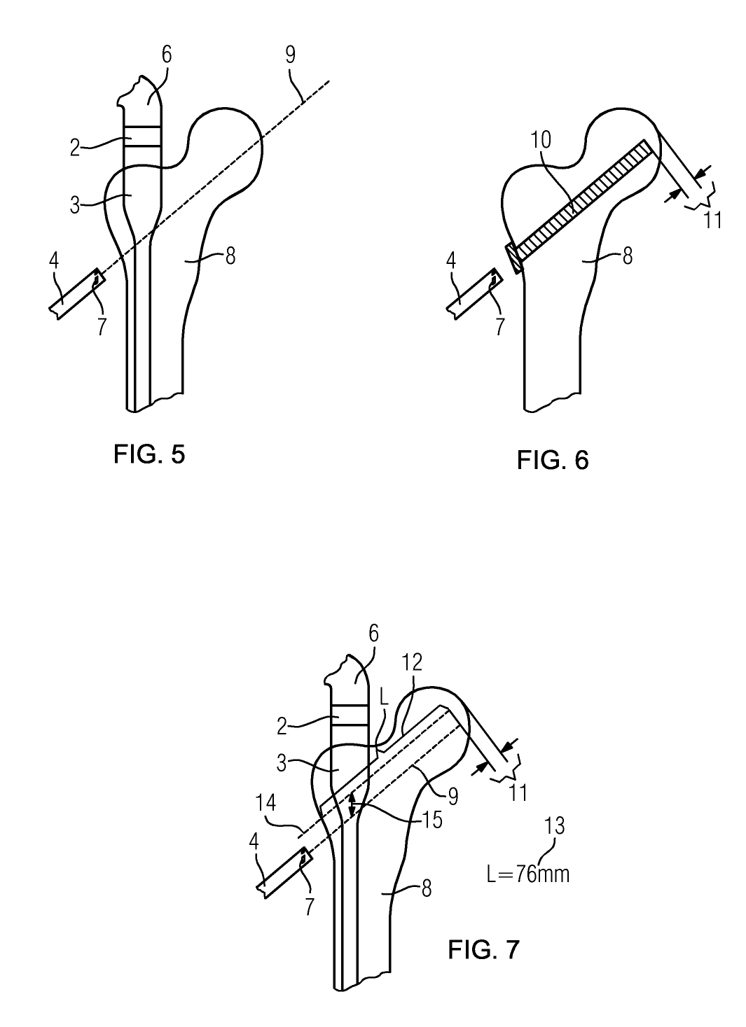

FIG. 5 shows an example for the superposed illustration of a projected straight line and an x-ray image. The x-ray image shown in FIG. 5 is identical to the x-ray image shown in FIG. 4. However, the projected straight line 9, which is calculated by the computer, has additionally been superposed onto this x-ray image. As a result of the superposed illustration of the projected straight line 9 with the x-ray image and, in particular, the bone 8, the user can immediately identify at which position a locking element would be introduced into the bone.

A multiplicity of additional illustrations are possible in the method according to the invention. An example of such an illustration is shown in FIG. 6. Since a bone model can be calculated within the scope of the method, a purely virtual illustration is possible. Using this, as shown in FIG. 6, it is possible to show only the bone 8, a virtual locking element 10, an additional information element 11 and a graphical illustration of the guide instrument 4 and of the x-ray markers 7. As a result, a clearer illustration is achieved. The illustration of the bone 8 is an image of the data from the bone model. The virtual locking element 10 is a locking element which is determined by the parameters which are predetermined by the position and orientation of the guide instrument 4. Since the data of both the virtual locking element and the bone model are stored in the computer, it is simultaneously possible also to display distance information 11. Since the shown illustration is completely calculated from 3D data, it can also be freely rotated and zoomed.

FIG. 7 shows an example for depicting a position correction as a superposed illustration of the x-ray image and further information. The guide apparatus 4 with the x-ray markers 7 arranged thereon, the target apparatus 6, and the intramedullary rod 3 and the bone 8 are imaged as an x-ray image. Additional information marked in color is superposed on this x-ray image. Thus, the projected straight line 9 is shown for the current position and orientation of the guide instrument. However, additionally, information relating to the position correction and parameters of the locking element to be introduced are also shown. The straight line 14 is an intended position, at which the introduction of a locking element would be advantageous. Arrow 15 shows to the user the direction in which and the extent to which the target apparatus should be displaced in the longitudinal direction of the bone. At the same time, the distance marking 11 shows a minimum distance from the bone wall and the length marking 12 and the associated alphanumerical display 13 indicate to the user that he should select a locking element with a length of 76 mm.

It is clear to a person skilled in the art that, in addition to the shown information, a plurality of further items of information or types of display can be selected. Therefore, it is not only possible to introduce locking elements with less damage to the bone structure and, possibly, with a reduction in the radiation exposure of the patient, but a user is also provided with additional information which can further improve the implantation result.

Although the invention is illustrated more closely and described in detail by the preferred exemplary embodiment, the invention is not restricted by the disclosed examples and other variations can be derived from this by a person skilled in the art, without departing from the scope of protection of the invention.

1. A method for visually assisting a user when fixing an implant by way of a locking element in or on a bone, the method comprising:
providing a guide instrument with at least one x-ray marker;
setting a location of the guide instrument for guiding the locking element in relation to the implant with a target apparatus, wherein the implant can be rotated and/or positioned by moving the target apparatus;
recording at least one x-ray image within a defined image region containing the target region of the locking element and the x-ray marker;
determining an orientation and a position of a projected straight line, which forms a projection of a longitudinal axis of the guide instrument in an image plane, from at least one of a position or a form of the image of the x-ray marker in the x-ray image using a computer;
calculating with the computer a superposed illustration of the x-ray image with a graphical element indicating the orientation and position of the projected straight line; and
outputting the superposed illustration on a display apparatus.

2. The method according to claim 1, which comprises recording at least two x-ray images with an offset relative to one another, either using different projection directions or parallel projection directions and the recording is perpendicular to the projection directions.

3. The method according to claim 1, which comprises calculating the position and the orientation of the longitudinal axis of the guide instrument by calculating an angle between the image plane of the x-ray image and the longitudinal axis and calculating a position of the longitudinal axis by taking into account at least one of the following: a known restriction in degrees of freedom of movement of the target apparatus, a form of the image of the x-ray marker in the x-ray image, a relative position of at least two x-ray markers in the x-ray image, and/or at least two x-ray images.

4. The method according to claim 3, which comprises, after recording the x-ray image, calculating a bone model with the computer using the x-ray image.

5. The method according to claim 4, which comprises generating the bone model from a parameterized model data record, which is adapted to the bone, or calculating the bone model by registering the x-ray image with an anatomical atlas.

6. The method according to claim 4, which comprises, after calculating the bone model and the position and orientation of the longitudinal axis, calculating at least one parameter of the locking element.

7. The method according to claim 6, which comprises selecting the locking element from a group of possible locking elements, and/or determining a length of the locking element, and/or calculating a position correction between a current position and an intended position of the target apparatus, wherein the position correction describes a rotation of the target apparatus about an axis extending through the bone and/or a shift between the target apparatus and the bone along the axis.

8. The method according to claim 6, which comprises, in addition to the superposed illustration, displaying an alphanumeric or graphical item of information representing at least one of the calculated parameter of the locking element or the position correction.

9. The method according to claim 6, which comprises, depending on the calculated position correction, providing to the user optical, acoustic or haptic notifications for correcting the position of the target apparatus.

10. The method according to claim 6, which comprises displaying a graphical element depicting the calculated parameter of the locking element by displaying an image of the locking element, which has this parameter, in the locking position.

11. The method according to claim 1, which comprises, after establishing the orientation and position of the projected straight line or of the longitudinal axis, continuously detecting the position and/or the orientation of the target apparatus or of the guide instrument by a second detection system, and continuously updating the orientation and the position of the projected straight line or of the longitudinal axis on the basis of the continuously detected information.

12. The method according to claim 11, wherein the second detection system is an optical detection system.

13. The method according to claim 1, wherein the x-ray marker is arranged on the end of the guide instrument facing the implant.

14. A target apparatus for assisting a user when fixing an implant by way of a locking element in or on a bone, the target apparatus comprising:
a fastening element for fastening the target apparatus to the implant in a spatially secured manner;
a guide instrument for guiding the locking element, said guide instrument having at least one x-ray marker; and
a connection element for coupling said fastening element and said guide instrument in a spatially secured manner.

15. The target apparatus according to claim 14, wherein said guide instrument is a tissue protection sleeve for protecting tissue when introducing said locking element.

16. The target apparatus according to claim 14, wherein said x-ray marker is disposed at an end of said guide instrument facing the implant.
